(19) United States
(12) Patent Application Publication
(10) Pub. No.: US 2025/0143870 A1
(43) Pub. Date: May 8, 2025

(54) ASTIGMATISM AXIS ADJUSTABLE ARTIFICIAL LENS

(71) Applicant: THE CATHOLIC UNIVERSITY OF KOREA INDUSTRY-ACADEMIC COOPERATION FOUNDATION, Seoul (KR)

(72) Inventor: Dong Hyun JEE, Seoul (KR)

(21) Appl. No.: 18/938,040

(22) Filed: Nov. 5, 2024

(30) Foreign Application Priority Data
Nov. 7, 2023 (KR) 10-2023-0152985

(51) Int. Cl. A61F 2/16

(52) U.S. Cl. CPC A61F 2/1624; A61F 2002/169; A61F 2002/16902

(57) ABSTRACT

An embodiment relates to an astigmatism axis adjustable artificial lens which is adjustable so that a correction axis of the artificial lens matches an astigmatism axis when the astigmatism axis and the correction axis of the artificial lens do not match. Here, the astigmatism axis adjustable artificial lens includes a support part and an optical lens part. The support part is fixed to a lens capsule of a patient's eye. The optical lens part is coupled to the support part and has a correction axis that matches the patient's astigmatism axis. The optical lens part is rotatably coupled to the support part to be adjustable so that the correction axis matches the patient's astigmatism axis.

### CROSS-REFERENCE TO RELATED APPLICATIONS

This application claims the priority of Korean Patent Application No. 10-2023-0152985 filed on Nov. 7, 2023, in the Korean Intellectual Property Office, the disclosure of which is incorporated herein by reference.

### BACKGROUND

The disclosure relates to an astigmatism axis adjustable artificial lens and, more specifically, to an astigmatism axis adjustable artificial lens which is adjustable so that a correction axis of the artificial lens matches an astigmatism axis when the astigmatism axis and the correction axis of the artificial lens do not match.

Cataract is a disease in which the lens becomes cloudy and vision deteriorates. Cataract is treated with surgery. Cataract surgery involves making a small 3 mm incision in the eye, inserting a device called an ultrasonic emulsifier, emulsifying (liquefying) the cataract, and suctioning it out. Thereafter, a soft artificial lens is rolled up and inserted into the eye.

Recently, astigmatism-correcting artificial lenses are widely used to correct astigmatism after cataract surgery. Astigmatism is a condition in which one side coming into the retina is distorted and refracted, and an artificial lens that corrects this is structured to create a refractive state opposite to the astigmatism state and offset the astigmatism of the eye when inserted correctly into the eye.

An astigmatism-correcting artificial lens has a function of offsetting a patient's astigmatism on the retina by refracting light entering the eye obliquely. Therefore, as for the astigmatism-correcting artificial lens, the patient's astigmatism axis must be found out through a preoperative examination, and then inserted to be aligned with that axis.

The most important thing at this time is to insert the axis of the astigmatism-correcting artificial lens, that is, the correction axis of the astigmatism-correcting artificial lens, in line with the patient's astigmatism axis. Only then can the patient's astigmatism be offset. If the axis of the astigmatism-correcting artificial lens and the axis of astigmatism are different, complications that worsen astigmatism may occur. Therefore, when using the astigmatism-correcting artificial lens, it is most important to align the axis.

Once an artificial lens is inserted into the eye, the degree cannot be adjusted. It would be good to try it on a patient's eye like fitting glasses and replace it with another lens if it doesn't fit, but it is virtually impossible to remove the artificial lens and replace it. Therefore, after the surgery is completed and a patient fully recovers, an artificial lens is checked at an outpatient clinic to see if it fits the patient's eye well.

The problem is that once an artificial lens is inserted, it cannot be replaced. Once inserted, a lens capsule and an artificial lens support part (Haptic) become adhered to each other and cannot move. Even if the astigmatism axes match in the surgical field of view, if a patient rubs his/her eye after surgery or is injured by a soccer ball or fist, the artificial lens astigmatism axis will turn. Otherwise, the axis of an artificial lens may turn for no reason. In rare cases, a patient's astigmatism itself may change. In cases like this, when the astigmatism axis changes after surgery, there is a problem that there is no way to correct it.

If it is confirmed that the astigmatism-correcting artificial lens does not fit well, a patient has to live with unclear vision or correct it with glasses. Even if the axis of the astigmatism-correcting artificial lens is not aligned, it is rare for it to be replaced. This is because the risks far outweigh the benefits.

Republic of Korea Patent Publication No. 2018-0090119 (Published on Aug. 10, 2018)

### SUMMARY

As aspect of the disclosure is to provide an astigmatism axis adjustable artificial lens which is adjustable so that a correction axis of the artificial lens matches an astigmatism axis when the astigmatism axis and the correction axis of the artificial lens do not match.

The aspect of the disclosure is not limited to that mentioned above, and other aspects not mentioned will be clearly understood by those skilled in the art from the description below.

An embodiment of the disclosure provides an astigmatism axis adjustable artificial lens, including: a support part fixed to a lens capsule of a patient's eye; and an optical lens part coupled to the support part and having a correction axis that matches the patient's astigmatism axis, wherein the optical lens part is rotatably coupled to the support part to be adjustable so that the correction axis matches the patient's astigmatism axis.

In an embodiment of the disclosure, the support part may have: a body having a mounting hole formed through the center thereof to which the optical lens part is mounted; a fixture connected to the outer circumferential surface of the body and fixed to a lens capsule of the patient's eye, and a guide groove formed along the circumferential direction of the mounting hole on the inner circumferential surface of the body.

In an embodiment of the disclosure, the optical lens part may have: an optical lens rotatably inserted into the mounting hole and having a correction axis; and a guide protrusion formed protruding on the outer circumferential surface of the optical lens and coupled to the guide groove so that the optical lens is rotated without being separated from the mounting hole.

In an embodiment of the disclosure, the guide protrusion may be formed continuously in the circumferential direction of the optical lens.

In an embodiment of the disclosure, the optical lens part may have first fitting grooves formed to be dented in the optical lens at a position that is misaligned with the central axis of the optical lens to allow a first lens hook to be fitted from the outside.

In an embodiment of the disclosure, the first fitting grooves may be formed as a pair symmetrically spaced based on the central axis of the optical lens.

In an embodiment of the disclosure, the optical lens part may have a first metal part provided in the first fitting grooves.

In an embodiment of the disclosure, the support part may further have a second fitting groove formed to be dented in at least one of the body and the fixture to allow a second lens hook to be fitted from the outside.

In an embodiment of the disclosure, the support part may have a second metal part provided in the second fitting groove.

According to an embodiment of the disclosure, an optical lens part may be independently rotatable while a support part is fixed. Accordingly, even if there occurs a situation where an astigmatism axis adjustable artificial lens is inserted into a patient's eye and the patient's astigmatism axis and a correction axis do not match after the support part is fixed by adhesion to a lens capsule, adjustment may be possible so that an optical lens is rotated to make the correction axis match the astigmatism axis.

The effects of the disclosure are not limited to the effects described above, and should be understood to include all effects that are inferable from the configuration of the disclosure described in the detailed description or claims of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is an exemplary view showing an astigmatism axis adjustable artificial lens according to an embodiment of the disclosure;

FIG. 2 is an exemplary view showing a support part of an astigmatism axis adjustable artificial lens according to an embodiment of the disclosure;

FIG. 3 is an exemplary cross-sectional view centered on an optical lens part of an astigmatism axis adjustable artificial lens according to an embodiment of the disclosure;

FIGS. 4A and 4B are each an exemplary plan view showing an optical lens part of an astigmatism axis adjustable artificial lens according to an embodiment of the disclosure;

FIG. 5 is an exemplary view explaining an example of using first fitting grooves, a second fitting groove, and a lens hook of an astigmatism axis adjustable artificial lens according to an embodiment of the disclosure;

FIGS. 6A and 6B are each an exemplary view explaining a method for matching an astigmatism axis and a correction axis of an optical lens part using the first fitting grooves, the second fitting groove, and the lens hook of FIG. 5;

FIG. 7 is a cross-sectional exemplary view of an optical lens part showing that a first metal part and a second metal part are provided in an astigmatism axis adjustable artificial lens, according to an embodiment of the disclosure;

FIGS. 8A and 8B are each an exemplary view explaining a method for matching an astigmatism axis and a correction axis of an optical lens part using the first metal part and second metal part of FIG. 7, and a magnet; and

FIG. 9 is a cross-sectional exemplary view of an optical lens part showing that a third metal part and a fourth metal part are provided in an astigmatism axis adjustable artificial lens, according to another embodiment of the disclosure.

### DETAILED DESCRIPTION

Hereinafter, the disclosure will be described with reference to the accompanying drawings. However, the disclosure may be implemented in various different forms, and therefore is not limited to the embodiments described herein. In addition, in order to clearly describe the disclosure in the drawings, parts that are not related to the description are omitted, and similar parts are given similar drawing reference numerals throughout the specification.

In the entire specification, when a part is said to be “connected (linked, contacted, coupled)” to another part, this includes not only the case where it is “directly connected” but also the case where it is “indirectly connected” with another member in between. In addition, when a part is said to “include” a certain component, this does not mean that other components are excluded unless otherwise specifically stated, but that other components may be additionally provided.

The terms used in this specification are used only to describe specific embodiments and are not intended to limit the disclosure. The singular expression includes the plural expression unless the context clearly indicates otherwise. In this specification, the terms “include” or “have” are intended to specify the presence of a feature, number, step, operation, component, part, or combination thereof described in the specification, but should be understood as not excluding in advance the possibility of the presence or addition of one or more other features, numbers, steps, operations, components, parts, or combinations thereof.

Hereinafter, embodiments of the disclosure will be described in detail with reference to the accompanying drawings.

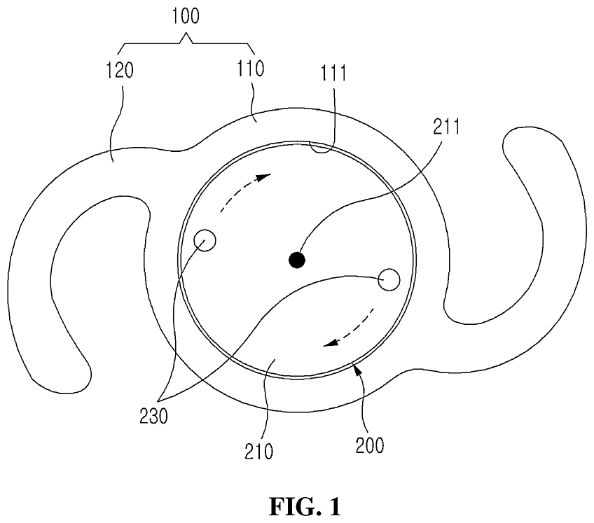

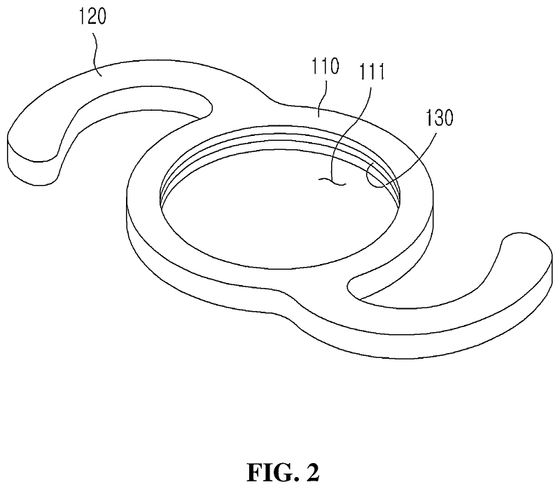

FIG. 1 is an exemplary view showing an astigmatism axis adjustable artificial lens according to an embodiment of the disclosure, and FIG. 2 is an exemplary view showing a support part of the astigmatism axis adjustable artificial lens according to an embodiment of the disclosure.

As shown in FIG. 1 and FIG. 2, an astigmatism correction axis adjustable artificial lens may include a support part (Haptic) 100 and an optical lens part (Optic) 200.

The support part 100 may be inserted through a small incision of about 3 mm made in a patient's eye.

The support part 100 may be made of flexible material, may be inserted through the incision in a rolled-up state, and may be stretched to return or unfold to its original shape after being inserted. The inserted support part 100 may then be fixed by adhesion to a lens capsule.

The support part 100 may have a body 110, a fixture 120, and a guide groove 130.

The body 110 may form the body of the support part 100. A mounting hole 111 may be formed through the center of the body 110, and an optical lens part 200 may be mounted in the mounting hole 111. The body 110 may be formed in a ring shape as a whole.

The fixture 120 may be connected to an outer circumferential surface of the body 110 and may be formed as a pair. The fixture 120 may be formed to extend to an outside of the body 110 and may be fixed to a lens capsule of a patient's eye.

The guide groove 130 may be formed along the circumferential direction of the mounting hole 111 on the inner circumferential surface of the body 110.

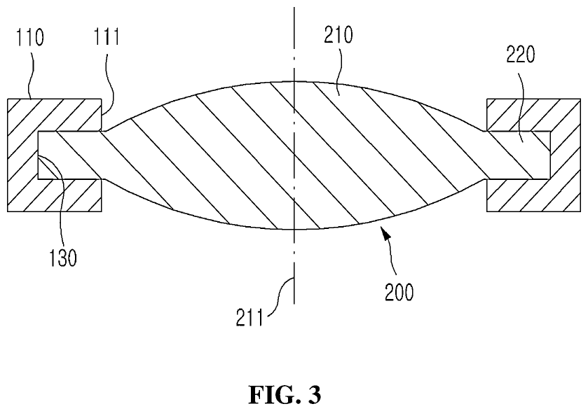

FIG. 3 is an exemplary cross-sectional view centered on an optical lens part of the astigmatism axis adjustable artificial lens according to an embodiment of the disclosure.

As shown in FIGS. 1 to 3, the optical lens part 200 may be coupled to the support part 100 and may have a correction axis that matches a patient's astigmatism axis.

Here, the optical lens part 200 may be rotatably coupled to the support part 100.

That is, the optical lens part 200 and the support part 100 are not formed as a single unit, but are formed separately to be separable from each other, and then may be coupled to each other.

In particular, the optical lens part 200 may be independently rotatable while the support part 100 is fixed. Through this, even if there occurs a situation where a patient's astigmatism axis and a correction axis do not match after the astigmatism axis adjustable artificial lens is inserted into the patient's eye and the support part 100 is adhered to and fixed with a lens capsule, adjustment may be possible and the optical lens part 200 is rotated to make the correction axis match the astigmatism axis.

Specifically, the optical lens part 200 may have an optical lens 210 and a guide protrusion 220.

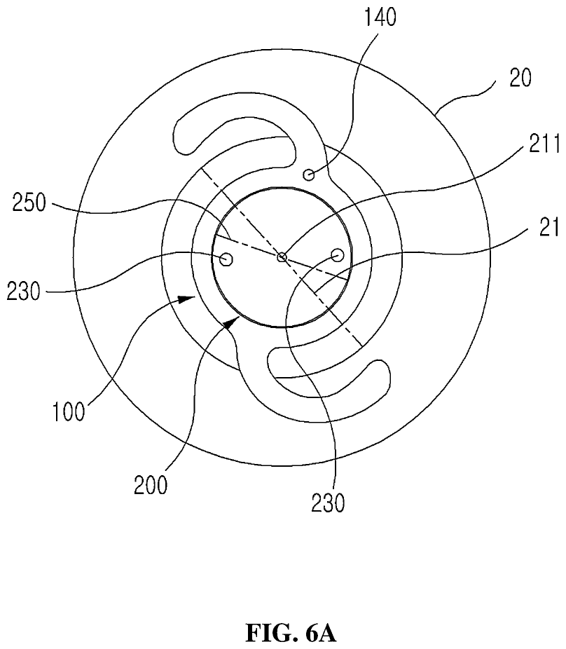

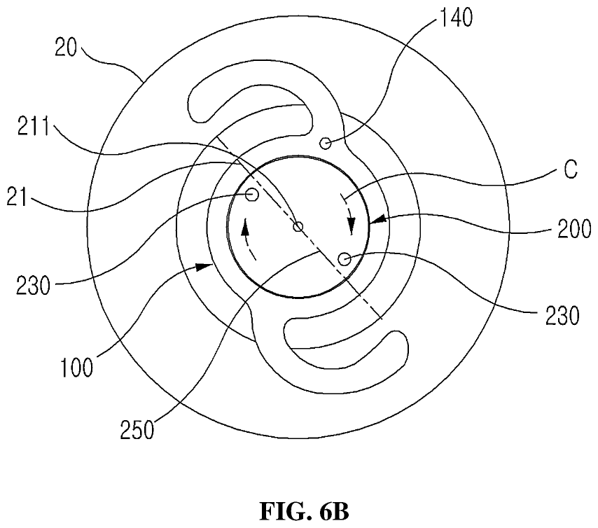

The optical lens 210 is a part through which light passes. The optical lens 210 may be rotatably inserted into and coupled to the mounting hole 111 and may have a correction axis 250 (see FIG. 6).

The guide protrusion 220 may be formed to protrude on the outer circumferential surface of the optical lens 210. When the optical lens 210 is inserted into the mounting hole 111, the guide protrusion 220 may be coupled to the guide groove 130.

The optical lens 210 may be rotatable in opposite directions based on a central axis 211. In addition, the guide protrusion 220 may be coupled to the guide groove 130 and restrained to prevent the optical lens 210 i from separating from the mounting hole 111. Accordingly, the optical lens 210 may be stably rotated without being separated from the mounting hole 111.

The guide protrusion 220 may be formed to be in close contact with the guide groove 130, and is preferable to be formed such that a frictional force is generated to prevent the optical lens 210 from rotating when no external force is applied.

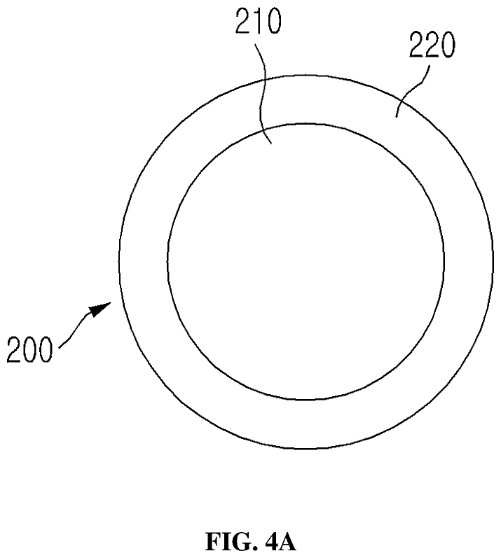

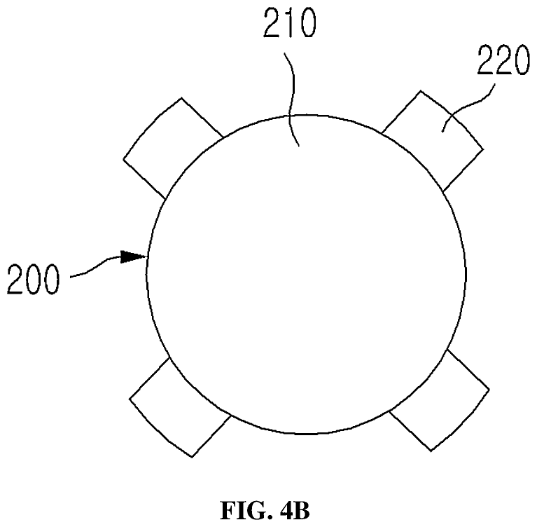

FIGS. 4A and 4B are each an exemplary plan view showing an optical lens part of the astigmatism axis adjustable artificial lens according to an embodiment of the disclosure.

As shown in FIG. 4A, the guide protrusion 220 may be formed continuously in the circumferential direction of the optical lens 210. In this form, since the guide protrusion 220 may be more securely inserted into the guide groove 130, the optical lens 210 may be more stably coupled to the mounting hole 111.

Alternatively, as shown in FIG. 4B, the guide protrusion 220 may be spaced at a certain interval along the circumferential direction of the optical lens 210. In this case, there is an advantage in that the size of the guide protrusion 220 can be reduced to make it lighter, and the amount of material required to form the guide protrusion 220 can be saved.

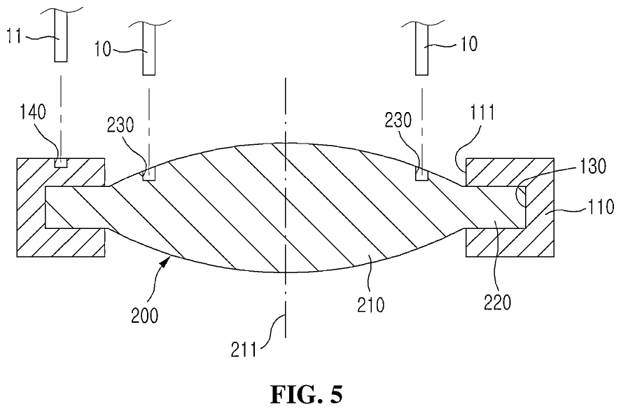

FIG. 5 is an exemplary view explaining an example of using first fitting grooves, a second fitting groove, and a lens hook of the astigmatism axis adjustable artificial lens according to an embodiment of the disclosure.

As shown in FIG. 1 and FIG. 5, the optical lens part 200 may have first fitting grooves 230.

The first fitting grooves 230 may be formed on the surface of the optical lens 210. The first fitting grooves 230 may be formed to be dented at a position that is misaligned with the central axis 211 of the optical lens 210.

A first lens hook 10 may be fitted into the first fitting grooves 230 from the outside. The first lens hook 10 is a rod-shaped member that may be fitted into the first fitting grooves 230. The first fitting grooves 230 are formed at a position that is misaligned with the central axis 211 of the optical lens 210, and thus, when a surgeon inserts the first lens hook 10 into the first fitting grooves 230 and rotates the first lens hook 10 around the central axis 211, the optical lens 210 may be rotatable around the central axis 211.

The first fitting grooves 230 may be formed as a pair symmetrically spaced apart based on the central axis 211 of the optical lens 210, and through this, the rotation of the optical lens 210 using the first lens hook 10 may be performed more effectively and stably.

In addition, the support part 100 may further have a second fitting groove 140 formed to be dented in at least one of the body 110 and the fixture 120 to allow a second lens hook 11 to be inserted from the outside.

FIGS. 6A and 6B are each an exemplary view explaining a method for matching an astigmatism axis and a correction axis of an optical lens part using the first fitting grooves, the second fitting groove, and the lens hook of FIG. 5.

As shown in FIG. 6, in a state where, while the astigmatism axis adjustable artificial lens is inserted into a patient's eye 20, a surgeon inserts the first lens hook 10 into the first fitting groove 230, inserts the second lens hook 11 into the second fitting groove 140, and then fixes the second lens hook 11 so that the support part 100 does not rotate, if the optical lens 210 is rotated (C) using the first lens hook 10, only the optical lens 210 may rotatable around the central axis 211, and through this, fine adjustment may be possible to align the correction axis 250 of the optical lens 210 with an astigmatism axis 21.

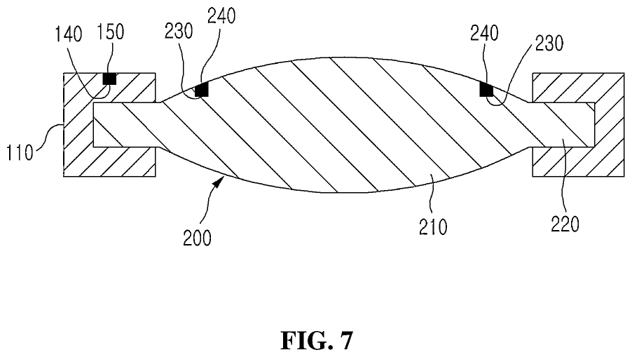

FIG. 7 is a cross-sectional exemplary view of an optical lens part showing that a first metal part and a second metal part are provided in the astigmatism axis adjustable artificial lens, according to an embodiment of the disclosure.

As shown in FIG. 7, the optical lens part 200 may have a first metal part 240. The first metal part 240 may be provided in the first fitting groove 230.

The first metal part 240 may be made of neodymium, which is known to have excellent biocompatibility even when inserted into the human body, nitinol, which is a 1:1 mixture of nickel and titanium, or stainless steel.

In addition, the support part 100 may have a second metal part 150, and the second metal part 150 may be provided in the second fitting groove 140. The second metal part 150 may be made of the same material as the first metal part 240.

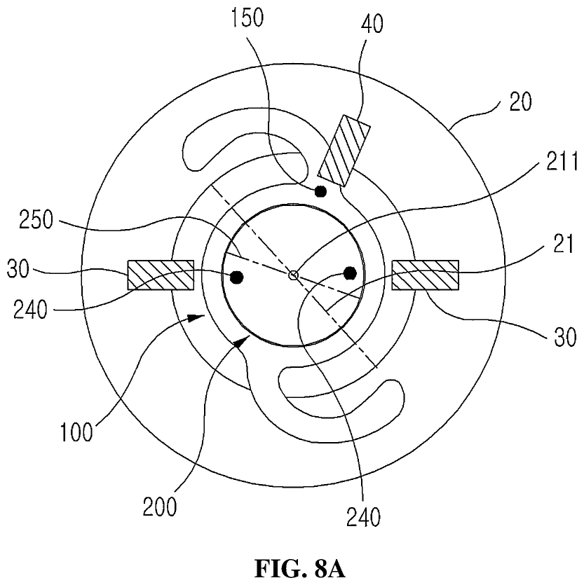

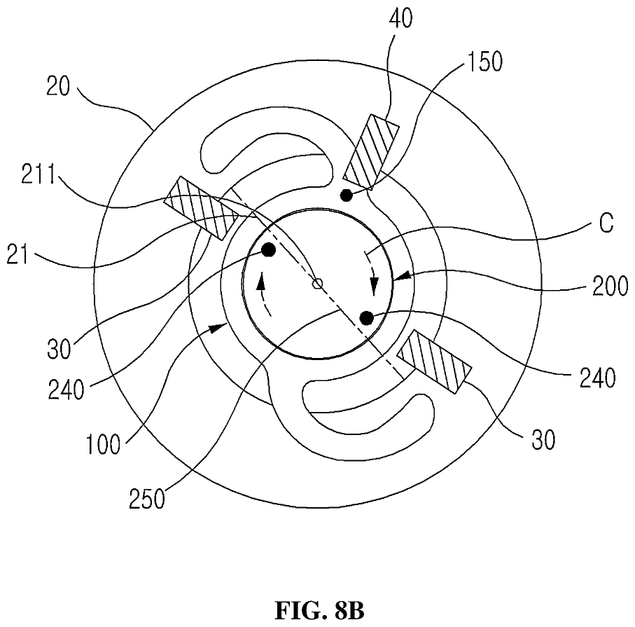

FIGS. 8A and 8B are each an exemplary view explaining a method for matching an astigmatism axis and a correction axis of an optical lens part using the first metal part and second metal part of FIG. 7, and a magnet.

As shown in FIGS. 8A and 8B, a surgeon may position a first magnet 30 near the first metal part 240 and position a second magnet 40 near the second metal part 150. Then, by fixing the second magnet 40 to fix the second metal part 150 and prevent the support part (100) from rotating, the first magnet 30 may be rotated to cause the first metal part 240 is rotated by the magnetic force, and as a result, the optical lens 210 may be rotated (C) around the central axis 211. Through this, fine rotation adjustment may be possible to align the correction axis 250 of the optical lens 210 with an astigmatism axis 21.

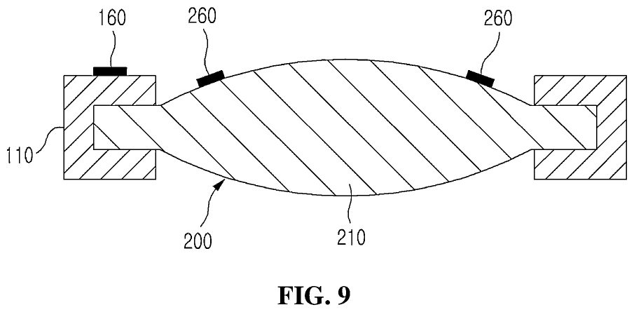

FIG. 9 is a cross-sectional exemplary view of an optical lens part showing that a third metal part and a fourth metal part are provided in the astigmatism axis adjustable artificial lens, according to another embodiment of the disclosure.

As shown in FIG. 9, the optical lens part 200 and the support part 100 may be provided in a different form from the first metal part and the second metal part described above. That is, referring to FIG. 7, although the first metal part 240 and the second metal part 150 were described above as being provided in the first fitting grooves 230 and the second fitting groove 140, respectively, the configuration of the first fitting grooves 230 and the second fitting groove 140 may be omitted and a metal part may be provided.

For example, as shown in FIG. 9, a third metal part 260 may be provided directly on the surface of the optical lens 210, and a fourth metal part 160 may be provided directly on the surface of the support part 100.

Meanwhile, the astigmatism axis adjustable artificial lens according to the disclosure is not limited to the form illustrated in FIG. 1, and of course, may also be applied to a toric lens, which is a lens for correcting astigmatism.

The description of the disclosure is for illustrative purposes, and those skilled in the art will understand that it can be easily modified into other specific forms without changing the technical idea or essential features of the disclosure. Therefore, the embodiments described above should be understood as being exemplary in all respects and not limiting. For example, each component described as a single type may be implemented in a distributed manner, and likewise, components described as distributed may be implemented in a combined form.

The scope of the disclosure is indicated by the following claims, and all changes or modifications derived from the meaning and scope of the claims and their equivalent concepts should be interpreted as being included in the scope of the disclosure.

[Explanation of reference numerals]

| | |
|---|---|
| 10: First lens hook | 11: Second lens hook |
| 21: Astigmatism axis | 30: First magnet |
| 40: Second magnet | 100: Support part |
| 110: Body | 111: Mounting hole |
| 120: Fixture | 130: Guide groove |
| 140: Second fitting groove | 150: Second metal part |
| 160: Fourth metal part | 200: Optical lens part |
| 210: Optical lens | 211: Central axis |
| 220: Guide protrusion | 230: First fitting groove |
| 240: First metal part | 250: Correction axis |
| 260: Third metal part | |

What is claimed is:

1. An astigmatism axis adjustable artificial lens, comprising:
a support part fixed to a lens capsule of an eye of a patient; and
an optical lens part coupled to the support part and having a correction axis that matches an astigmatism axis of the patient,
wherein the optical lens part is rotatably coupled to the support part to be adjustable so as to match the correction axis with the astigmatism axis of the patient.

2. The astigmatism axis adjustable artificial lens of claim 1, wherein the support part comprises:
a body having a mounting hole formed through a center thereof to which the optical lens part is mounted;
a fixture connected to an outer circumferential surface of the body and fixed to the lens capsule of the eye of the patient; and
a guide groove formed along a circumferential direction of the mounting hole on an inner circumferential surface of the body.

3. The astigmatism axis adjustable artificial lens of claim 2, wherein the optical lens part comprises:
an optical lens rotatably inserted into the mounting hole and having the correction axis; and
a guide protrusion, protruding on the outer circumferential surface of the optical lens and coupled to the guide groove, configured to rotate the optical lens without being separated from the mounting hole.

4. The astigmatism axis adjustable artificial lens of claim 3, wherein the guide protrusion is defined continuously in the circumferential direction of the optical lens.

5. The astigmatism axis adjustable artificial lens of claim 3, wherein the optical lens part includes first fitting grooves dented in the optical lens at a position that is misaligned with a central axis of the optical lens to allow a first lens hook to be fitted from an outside.

6. The astigmatism axis adjustable artificial lens of claim 5, wherein the first fitting grooves are positioned as a symmetrically spaced pair based on the central axis of the optical lens.

7. The astigmatism axis adjustable artificial lens of claim 5, wherein the optical lens part has a first metal part disposed in the first fitting grooves.

8. The astigmatism axis adjustable artificial lens of claim 5, wherein the support part further comprises a second fitting groove formed to be dented in at least one of the body and the fixture to allow a second lens hook to be fitted from the outside.

9. The astigmatism axis adjustable artificial lens of claim 8, wherein the support part further comprises a second metal part disposed in the second fitting groove.
